(19) United States
(12) Patent Application Publication
(10) Pub. No.: US 2011/0319492 A1
(43) Pub. Date: Dec. 29, 2011

(54) MANUFACTURE OF TABLETS FROM ENERGY-APPLIED POWDER BLEND

(76) Inventors: Joseph R. Luber, Quakertown, PA (US); Christopher E. Szymczak, Marlton, NJ (US); Frank J. Bunick, Randolph, NJ (US); Harry S. Sowden, Glenside, PA (US); Leo B. Kriksunov, Ithaca, NY (US)

(21) Appl. No.: 13/052,200

(22) Filed: Mar. 21, 2011

Related U.S. Application Data
(60) Provisional application No. 61/358,167, filed on Jun. 24, 2010.

(51) Int. Cl. A61K 31/192; H05B 6/00; A61K 31/167

(52) U.S. Cl. 514/570; 514/630; 264/460

(57) ABSTRACT

The present invention features a process for making tablets containing one or more pharmaceutically active agent(s) and one or more binder(s), the method including the steps of (i) applying energy to a powder blend comprising the pharmaceutically active agent(s) and the binder(s) for a sufficient period of time to activate the binder(s) within the powder blend and (ii) forming a predetermined amount of the energy-applied powder blend into the tablets.

### CROSS REFERENCE TO RELATED APPLICATIONS

This application claims priority of the benefits of the filing of U.S. Provisional Application Ser. No. 61/245,315, filed Sep. 24, 2009, U.S. Provisional Application Ser. No. 61/255,582, filed Oct. 28, 2009, U.S. Provisional Application Ser. No. 61/314,629, filed Mar. 17, 2010, U.S. Provisional Application Ser. No. 61/358,167, filed Jun. 24, 2010, U.S. patent application Ser. No. 12/887,544, filed Sep. 22, 2010, and U.S. patent application Ser. No. 12/887,552, filed Sep. 22, 2010. The complete disclosures of the aforementioned related U.S. patent applications are hereby incorporated herein by reference for all purposes.

### BACKGROUND OF THE INVENTION

Pharmaceuticals intended for oral administration are typically provided in tablet form. Tablets are swallowed whole, chewed in the mouth, or disintegrated in the oral cavity. Soft tablets that either are chewed or dissolve in the mouth are often employed in the administration of pharmaceuticals where it is impractical to provide a tablet for swallowing whole. With chewable tablets, the act of chewing helps to break up the tablet particles as the tablet disintegrates and may increase the rate of absorption by the digestive tract. Soft tablets are also advantageous where it is desirable to make a pharmaceutically active agent available topically in the mouth or throat for both local effects and/or systemic absorption. Soft tablets are also utilized to improve drug administration in pediatric and geriatric patients. Soft tablets designed to disintegrate in the mouth prior to swallowing are particularly useful for improving compliance of pediatric patients.

Generally, soft tablets are made by compaction of a blend of powdered ingredients and typically include a pharmaceutically active agent, flavoring, and/or binders. The powder blend is typically fed into the cavity of a die of a tablet press and a tablet is formed by applying pressure. Hardness of the resulting tablet is a direct function of the compaction pressure employed and the compatibility of the ingredients in the formulation. A softer tablet, having an easier bite-through, may be prepared by employing reduced compaction pressures. The resulting tablet is softer, but also more fragile, brittle, and easily chipped and disadvantageously can involve complex and costly processing steps. Examples of soft tablets designed to disintegrate in the mouth without chewing are disclosed in U.S. Pat. Nos. 5,464,632, 5,223,264, 5,178,878, 6,589,554, and 6,224,905.

Swallowable tablets have been produced utilizing a melt extrusion process, where the active ingredient is mixed with excipients, heated as a mass and extruded into preformed dies. These tablets are intended to be directly swallowed, and in some cases, have modified or sustained release properties. Examples of melt extruded tablets are disclosed in U.S. Pat. Nos. 6,387,401 and 7,022,344.

There is a need for aesthetically pleasing chewable and orally disintegrating tablets that utilizes compression-based tableting machines typically used to produce high density, hard swallowable tablets. When used at low compression forces, these machines typically produce highly friable tablets, which are not sufficiently stable during packaging, shipping, and storage. The present invention relates to the discovery of a process for making tablets, such as chewable or orally disintegrating tablets, by applying energy (e.g., radiofrequency energy) to a powder blend to activate a binder within said powder blend.

### SUMMARY OF THE INVENTION

In one aspect, the present invention features a process for making tablets containing one or more pharmaceutically active agent(s) and one or more binder(s), the method including the steps of (i) applying energy to a powder blend comprising the pharmaceutically active agent(s) and the binder(s) for a sufficient period of time to activate the binder(s) within the powder blend and (ii) forming a predetermined amount of the energy-applied powder blend into the tablets. In a further embodiment, the tablets have a density less than 0.8 g/cc and the tablets disintegrate in the mouth when placed on the tongue in less than about 30 seconds.

In another aspect, the present invention features a process for making tablets by applying radiofrequency energy to a powder blend containing a pharmaceutically active agent and a RF-meltable binder for a sufficient period of time to soften or melt said RF-binder and forming a predetermined amount of the energy-applied powder blend into the tablets.

In another aspect, the present invention features a tablet manufactured according to the above processes.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments can be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

As discussed above, in one aspect, the present invention features a process for making tablets containing one or more pharmaceutically active agent(s) and one or more binder(s), the method including the steps of (i) applying energy to a powder blend comprising the pharmaceutically active agent(s) and the binder(s) for a sufficient period of time to activate the binder(s) within the powder blend and (ii) forming a predetermined amount of the energy-applied powder blend into the tablets.

### Powder Blend

As discussed above, the tablet is manufactured by applying energy to a powder blend containing a pharmaceutically active agent (as discussed herein), a binder (as discussed herein), and optionally a pharmaceutically-acceptable carrier. Examples of binders include but are not limited to meltable binders and water-activated binding materials. The carrier contains one or more suitable excipients for the formulation of tablets. Examples of suitable excipients include, but are not limited to, fillers, adsorbents, disintegrants, lubricants, glidants, sweeteners, superdisintegrants, flavor and aroma agents, antioxidants, preservatives, texture enhancers, and mixtures thereof. One or more of the above ingredients may be present on the same particle of the powder blend.

Suitable fillers include, but are not limited to, carbohydrates (as discussed herein) and water insoluble plastically deforming materials (e.g., microcrystalline cellulose or other cellulosic derivatives), and mixtures thereof.

Suitable adsorbents include, but are not limited to, water-insoluble adsorbents such as dicalcium phosphate, tricalcium phosphate, silicified microcrystalline cellulose (e.g., such as distributed under the PROSOLV brand (PenWest Pharmaceuticals, Patterson, N.Y.)), magnesium aluminometasilicate (e.g., such as distributed under the NEUSILIN brand (Fuji Chemical Industries (USA) Inc., Robbinsville, N.J.)), clays, silicas, bentonite, zeolites, magnesium silicates, hydrotalcite, veegum, and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof.

Suitable glidants include, but are not limited to, colloidal silicon dioxide.

Examples of sweeteners include, but are not limited to, synthetic or natural sugars; artificial sweeteners such as saccharin, sodium saccharin, aspartame, acesulfame, thaumatin, glycyrrhizin, sucralose, dihydrochalcone, alitame, miraculin, monellin, and stevside; sugar alcohols such as sorbitol, mannitol, glycerol, lactitol, maltitol, and xylitol; sugars extracted from sugar cane and sugar beet (sucrose), dextrose (also called glucose), fructose (also called laevulose), and lactose (also called milk sugar); isomalt, salts thereof, and mixtures thereof.

Examples of superdisintegrants include, but are not limited to, croscarmellose sodium, sodium starch glycolate and cross-linked povidone (crospovidone). In one embodiment the tablet contains up to about 5% by weight of such superdisintegrant.

Examples of flavors and aromatics include, but are not limited to, essential oils including distillations, solvent extractions, or cold expressions of chopped flowers, leaves, peel or pulped whole fruit containing mixtures of alcohols, esters, aldehydes and lactones; essences including either diluted solutions of essential oils, or mixtures of synthetic chemicals blended to match the natural flavor of the fruit (e.g., strawberry, raspberry and black currant); artificial and natural flavors of brews and liquors, e.g., cognac, whisky, rum, gin, sherry, port, and wine; tobacco, coffee, tea, cocoa, and mint; fruit juices including expelled juice from washed, scrubbed fruits such as lemon, orange, and lime; spear mint, pepper mint, wintergreen, cinnamon, cacoe/cocoa, vanilla, liquorice, menthol, eucalyptus, aniseeds nuts (e.g., peanuts, coconuts, hazelnuts, chestnuts, walnuts, colanuts), almonds, raisins; and powder, flour, or vegetable material parts including tobacco plant parts, e.g., genus Nicotiana, in amounts not contributing significantly to the level of nicotine, and ginger.

Examples of antioxidants include, but are not limited to, tocopherols, ascorbic acid, sodium pyrosulfite, butylhydroxytoluene, butylated hydroxyanisole, edetic acid, and edetate salts, and mixtures thereof.

Examples of preservatives include, but are not limited to, citric acid, tartaric acid, lactic acid, malic acid, acetic acid, benzoic acid, and sorbic acid, and mixtures thereof.

Examples of texture enhancers include, but are not limited to, pectin, polyethylene oxide, and carrageenan, and mixtures thereof. In one embodiment, texture enhancers are used at levels of from about 0.1% to about 10% percent by weight.

In one embodiment of the invention, the powder blend has an average particle size of less than 500 microns, such as from about 50 microns to about 500 microns, such as from about 50 microns and 300 microns. Particles in this size range are particularly useful for direct compacting processes.

In one embodiment of the invention, the tablet may be a made from a powder blend that is substantially free of hydrated polymers. As used herein, what is meant by “substantially free” is less than 5%, such as less than 1%, such as less than 0.1%, such as completely free (e.g., 0%). Such a composition is advantageous for maintaining an immediate release dissolution profile, minimizing processing and material costs, and providing for optimal physical and chemical stability of the tablet.

In one embodiment, powder blend/tablet is substantially free of directly compressible water insoluble fillers. Water insoluble fillers include but are not limited to microcrystalline cellulose, directly compressible microcrystalline cellulose, celluloses, water insoluble celluloses, starch, cornstarch and modified starches. As described in this embodiment, substantially free is less than 2 percent, e.g. less than 1 percent or none.

### Meltable Binder

In one embodiment, the powder blend/tablet of the present invention includes at least one meltable binder. In one embodiment, the meltable binder has a melting point of from about 40° C. to about 140° C., such as from about 55° C. to about 100° C. The softening or melting of the meltable binder(s) results in the sintering of the powder blend through the binding of the softened or melted binder with the pharmaceutically active agent and/or other ingredients within the powder blend.

In one embodiment, the meltable binder is a RF-meltable binder. What is meant by an RF-meltable binder is a solid binder that can be softened or melted upon exposure to RF energy. The RF-meltable binder typically is polar and has the capability to re-harden or resolidify upon cooling.

In one embodiment, the meltable binder is not a RF-meltable binder. In such embodiment, the powder blend contains an excipient that heats upon exposure to RF energy (e.g., a polar excipient), such that the resulting heat from is able to soften or melt the meltable binder. Examples of such excipients include, but are not limited to, polar liquids such as water and glycerin; powdered metals and metal salts such as powdered iron, sodium chloride, aluminum hydroxide, and magnesium hydroxide; stearic acid maltodextrin and sodium stearate.

Other examples of meltable binders include amorphous carbohydrate polymers. What is meant by an “amorphous carbohydrate polymer” is a molecule having a plurality of carbohydrate monomers wherein such molecule has a crystallinity of less than 20%, such as less than 10%, such as less than 5%. Examples of amorphous carbohydrate polymers include, but are not limited to hydrogenated starch hydrosolate, polydextrose, and oligosaccharides. Examples of oligosaccharides include, but are not limited to, fructo-oligosaccharide, galacto-oligosaccharide malto-oligosaccharide, inulin, and isolmalto-oligosaccharide

Examples of suitable meltable binders include: fats such as cocoa butter, hydrogenated vegetable oil such as palm kernel oil, cottonseed oil, sunflower oil, and soybean oil; mono, di, and triglycerides; phospholipids; cetyl alcohol; waxes such as Carnauba wax, spermaceti wax, beeswax, candelilla wax, shellac wax, microcrystalline wax, glyceryl behenate, glycerol distearate, co-polymer of polyethylene glycol and propylene glycol, polyethylene glycol behenate and paraffin wax; water soluble polymers such as polyethylene glycol, polycaprolactone, GlycoWax-932, lauroyl macrogol-32 glycerides, and stearoyl macrogol-32 glycerides; polyethylene oxides; and sucrose esters.

In one embodiment, the meltable binder is a RF-meltable binder, and the RF-meltable binder is a polyethylene glycol (PEG), such as PEG-4000. A particularly preferred RF-meltable binder is PEG having at least 95% by weight of the PEG particles less than 100 microns (as measured by conventional means such as light or laser scattering or sieve analysis) and a molecular weight between 3000 and 8000 Daltons.

The meltable binder(s) may be present at level of about 0.01 percent to about 70 percent of the powder blend/tablet, such as from about 1 percent to about 50 percent, such as from about 10 percent to about 30 percent of the powder blend/tablet.

In one embodiment, the average particle size of the binder is less than 250 microns, such as less than 100 microns.

### Water-Containing Material

In one embodiment, the powder blend/tablet of the present invention includes at least one water-containing material. Examples of water-containing materials include, but are not limited to, materials wherein the water is chemically bound to the material (e.g., a hydrate salt), materials wherein the water is adsorbed or absorbed to the material (e.g., porous material such a silicas and microsponges), and materials that have water encapsulated therein (e.g., liquid filled capsules). Examples of such materials include, but are not limited to: fumed silicas; colloidal silicas such as colloidal silicon dioxide; silicates such as calcium silicate, aluminum silicate, magnesium aluminum metasilicate (such as NEUSILIN, US-2 from Fuji Chemical Ltd), and magnesium silicate; clays; zeolites; and veegum.

In one embodiment, the powder blend/tablet contains at least one hydrated salt. Examples of hydrated salts include, but are not limited to, sodium sulfate hydrate, sodium carbonate hydrate, calcium chloride hydrate, sodium hydrogen phosphate hydrate, and mixtures thereof. In one embodiment, the hydrated salt has molecular weight from about 150 to about 400 Daltons, such as from about 200 to about 350 Daltons.

In one embodiment, the powder blend/tablet contains at least one liquid filled capsule. In a further embodiment, the water is released from the capsule upon rupture, wherein such rupture is caused by the addition of energy.

The water-containing material(s) may be present at level of about 0.01 percent to about 70 percent of the powder blend/tablet, such as from about 1 percent to about 50 percent, such as from about 1 percent to about 30 percent, such as from about 2 percent to about 10 percent of the powder blend/tablet.

### Water-Activating Binding Material

In one embodiment, the powder blend/tablet of the present invention includes at least one water-activating binding material. What is meant by a water-activating binding material is a material that will activate or hydrate upon contact with water (e.g. released from the water containing material upon the addition of the energy) and assist in binding/fusing the powder blend into a tablet. Examples of such materials include, but are not limited to, hydrolyzed proteins, hydrating polymers and hydrocolloids. Suitable hydrolyzed proteins include, but are not limited to, hydrolyzed collagen. Suitable hydrating polymers include, but are not limited to starches, modified starches, methylcellulose, hydroxypropylcellulose, and hydroxypropylcellulose. Suitable hydrocolloids include, but are not limited to, gelatin, gellan gum, carrageenan, and pectin.

### Carbohydrates

In one embodiment, the powder blend contains at least one carbohydrate. The carbohydrate can contribute to the dissolvability and mouth feel of the tablet, aid in distributing the meltable binder across a broader surface area, and diluting and cushioning the pharmaceutically active agent. Examples of carbohydrates include, but are not limited to, water-soluble compressible carbohydrates such as sugars (e.g., dextrose, sucrose, maltose, isomalt, and lactose), starches (e.g., corn starch), sugar-alcohols (e.g., mannitol, sorbitol, maltitol, erythritol, lactitol, and xylitol), and starch hydrolysates (e.g., dextrins, and maltodextrins).

The carbohydrate(s) may be present at level of about 5 percent to about 95 percent of the powder blend/tablet, such as from about 20 percent to about 90 percent or from about 40 percent to about 80 percent of the powder blend/tablet. The particle size of the of carbohydrate can influence the level of meltable binder used, wherein a higher particle size of carbohydrate provides a lower surface area and subsequently requires a lower level of meltable binder. In one embodiment, wherein the carbohydrate(s) is greater than 50% by weight of the powder blend and the mean particle size of the carbohydrate(s) is greater than 100 microns, then the binder is from about 10 to about 30 percent by weight of the powder blend/tablet.

### Pharmaceutically Active Agent

The powder blend/tablet of the present invention includes at least one pharmaceutically active agent. What is meant by a “pharmaceutically active agent” is an agent (e.g., a compound) that is permitted or approved by the U.S. Food and Drug Administration, European Medicines Agency, or any successor entity thereof, for the oral treatment of a condition or disease. Suitable pharmaceutically active agents include, but are not limited to, analgesics, anti-inflammatory agents, antipyretics, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, nicotine, and sedatives.

Examples of suitable gastrointestinal treating agents include, but are not limited to: antacids such as aluminum-containing pharmaceutically active agents (e.g., aluminum carbonate, aluminum hydroxide, dihydroxyaluminum sodium carbonate, and aluminum phosphate), bicarbonate-containing pharmaceutically active agents, bismuth-containing pharmaceutically active agents (e.g., bismuth aluminate, bismuth carbonate, bismuth subcarbonate, bismuth subgallate, and bismuth subnitrate), calcium-containing pharmaceutically active agents (e.g., calcium carbonate), glycine, magnesium-containing pharmaceutically active agents (e.g., magaldrate, magnesium aluminosilicates, magnesium carbonate, magnesium glycinate, magnesium hydroxide, magnesium oxide, and magnesium trisilicate), phosphate-containing pharmaceutically active agents (e.g., aluminum phosphate and calcium phosphate), potassium-containing pharmaceutically active agents (e.g., potassium bicarbonate), sodium-containing pharmaceutically active agents (e.g., sodium bicarbonate), and silicates; laxatives such as stool softeners (e.g., docusate) and stimulant laxatives (e.g., bisacodyl); H2 receptor antagonists, such as famotidine, ranitidine, cimetadine, and nizatidine; proton pump inhibitors such as omeprazole, dextansoprazole, esomeprazole, pantoprazole, rabeprazole, and lansoprazole; gastrointestinal cytoprotectives, such as sucraflate and misoprostol; gastrointestinal prokinetics such as prucalopride; antibiotics for H. pylori, such as clarithromycin, amoxicillin, tetracycline, and metronidazole; antidiarrheals, such as bismuth subsalicylate, kaolin, diphenoxylate, and loperamide; glycopyrrolate; analgesics, such as mesalamine; antiemetics such as ondansetron, cyclizine, diphenyhydroamine, dimenhydrinate, meclizine, promethazine, and hydroxyzine; probiotic bacteria including but not limited to lactobacilli; lactase; racecadotril; and antiflatulents such as polydimethylsiloxanes (e.g., dimethicone and simethicone, including those disclosed in U.S. Pat. Nos. 4,906,478, 5,275,822, and 6,103,260); isomers thereof; and pharmaceutically acceptable salts and prodrugs (e.g., esters) thereof.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof; and pharmaceutically acceptable salts and prodrugs thereof.

Examples of muscle relaxants include, but are not limited to, cyclobenzaprine and chlorzoxazone metaxalone, orphenadrine, and methocarbamol; isomers thereof; and pharmaceutically acceptable salts and prodrugs thereof.

Examples of stimulants include, but are not limited to, caffeine.

Examples of sedatives include, but are not limited to sleep aids such as antihistamines (e.g., diphenhydramine), eszopiclone, and zolpidem, and pharmaceutically acceptable salts and prodrugs thereof.

Examples of appetite suppressants include, but are not limited to, phenylpropanolamine, phentermine, and diethylcathinone, and pharmaceutically acceptable salts and prodrugs thereof.

Examples of anesthetics (e.g., for the treatment of sore throat) include, but are not limited to dyclonine, benzocaine, and pectin and pharmaceutically acceptable salts and prodrugs thereof.

Examples of suitable statins include but are not limited to atorvastin, rosuvastatin, fluvastatin, lovastatin, simvustatin, atorvastatin, pravastatin and pharmaceutically acceptable salts and prodrugs thereof.

In one embodiment, the pharmaceutically active agent included within the tablet is selected from phenylephrine, dextromethorphan, pseudoephedrine, acetaminophen, cetirizine, aspirin, nicotine, ranitidine, ibuprofen, ketoprofen, loperamide, famotidine, calcium carbonate, simethicone, chlorpheniramine, methocarbomal, chlophedianol, ascorbic acid, pectin, dyclonine, benzocaine and menthol, and pharmaceutically acceptable salts and prodrugs thereof.

As discussed above, the pharmaceutically active agents of the present invention may also be present in the form of pharmaceutically acceptable salts, such as acidic/anionic or basic/cationic salts. Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Pharmaceutically acceptable basic/cationic salts include, and are not limited to aluminum, benzathine, calcium, chloroprocaine, choline, diethanolamine, ethylenediamine, lithium, magnesium, meglumine, potassium, procaine, sodium and zinc.

As discussed above, the pharmaceutically active agents of the present invention may also be present in the form of prodrugs of the pharmaceutically active agents. In general, such prodrugs will be functional derivatives of the pharmaceutically active agent, which are readily convertible in vivo into the required pharmaceutically active agent. Conventional procedures for the selection and preparation of suitable prodrug derivatives are described, for example, in “Design of Prodrugs”, ed. H. Bundgaard, Elsevier, 1985. In addition to salts, the invention provides the esters, amides, and other protected or derivatized forms of the described compounds.

Where the pharmaceutically active agents according to this invention have at least one chiral center, they may accordingly exist as enantiomers. Where the pharmaceutically active agents possess two or more chiral centers, they may additionally exist as diastereomers. It is to be understood that all such isomers and mixtures thereof are encompassed within the scope of the present invention. Furthermore, some of the crystalline forms for the pharmaceutically active agents may exist as polymorphs and as such are intended to be included in the present invention. In addition, some of the pharmaceutically active agents may form solvates with water (e.g., hydrates) or common organic solvents, and such solvates are also intended to be encompassed within the scope of this invention.

In one embodiment, the pharmaceutically active agent or agents are present in the tablet in a therapeutically effective amount, which is an amount that produces the desired therapeutic response upon oral administration and can be readily determined by one skilled in the art. In determining such amounts, the particular pharmaceutically active agent being administered, the bioavailability characteristics of the pharmaceutically active agent, the dose regime, the age and weight of the patient, and other factors must be considered, as known in the art.

The pharmaceutically active agent may be present in various forms. For example, the pharmaceutically active agent may be dispersed at the molecular level, e.g. melted, within the tablet, or may be in the form of particles, which in turn may be coated or uncoated. If the pharmaceutically active agent is in form of particles, the particles (whether coated or uncoated) typically have an average particle size of from about 1 to about 2000 microns. In one embodiment, such particles are crystals having an average particle size of from about 1 to about 300 microns. In another embodiment, the particles are granules or pellets having an average particle size of from about 50 to about 2000 microns, such as from about 50 to about 1000 microns, such as from about 100 to about 800 microns.

The pharmaceutically active agent may be present in pure crystal form or in a granulated form prior to the addition of the taste masking coating. Granulation techniques may be used to improve the flow characteristics or particle size of the pharmaceutically active agents to make it more suitable for compaction or subsequent coating. Suitable binders for making the granulation include but are not limited to starch, polyvinylpyrrolidone, polymethacrylates, hydroxypropylmethylcellulose, and hydroxypropylcellulose. The particles including pharmaceutically active agent(s) may be made by cogranulating the pharmaceutically active agent(s) with suitable substrate particles via any of the granulation methods known in the art. Examples of such granulation method include, but are not limited to, high sheer wet granulation and fluid bed granulation such as rotary fluid bed granulation.

If the pharmaceutically active agent has an objectionable taste, the pharmaceutically active agent may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked pharmaceutically active agents may also be employed. For example, acetaminophen particles, which are encapsulated with ethylcellulose or other polymers by a coacervation process, may be used in the present invention. Coacervation-encapsulated acetaminophen may be purchased commercially from Eurand America, Inc. (Vandalia, Ohio) or from Circa Inc. (Dayton, Ohio).

In one embodiment, the tablet incorporates modified release coated particles (e.g., particles containing at least one pharmaceutically active agent that convey modified release properties of such agent). As used herein, “modified release” shall apply to the altered release or dissolution of the active agent in a dissolution medium, such as gastrointestinal fluids. Types of modified release include, but are not limited to, sustained release or delayed release. In general, modified release tablets are formulated to make the active agents(s) available over an extended period of time after ingestion, which thereby allows for a reduction in dosing frequency compared to the dosing of the same active agent(s) in a conventional tablet. Modified release tablets also permit the use of active agent combinations wherein the duration of one pharmaceutically active agent may differ from the duration of another pharmaceutically active agent. In one embodiment the tablet contains one pharmaceutically active agent that is released in an immediate release manner and an additional active agent or a second portion of the same active agent as the first that is modified release.

Examples of swellable, erodible hydrophilic materials for use as a release modifying excipient for use in the modified release coating include water swellable cellulose derivatives, polyalkylene glycols, thermoplastic polyalkylene oxides, acrylic polymers, hydrocolloids, clays, and gelling starches. Examples of water swellable cellulose derivatives include sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose. Examples of polyalkylene glycols include polyethylene glycol. Examples of suitable thermoplastic polyalkylene oxides include poly(ethylene oxide). Examples of acrylic polymers include potassium methacrylatedivinylbenzene copolymer, polymethylmethacrylate, and high-molecular weight cross-linked acrylic acid homopolymers and copolymers.

Suitable pH-dependent polymers for use as release-modifying excipients for use in the modified release coating include: enteric cellulose derivatives such as hydroxypropyl methylcellulose phthalate, hydroxypropyl methylcellulose acetate succinate, and cellulose acetate phthalate; natural resins such as shellac and zein; enteric acetate derivatives such as polyvinylacetate phthalate, cellulose acetate phthalate, and acetaldehyde dimethylcellulose acetate; and enteric acrylate derivatives such as for example polymethacrylate-based polymers such as poly(methacrylic acid, methyl methacrylate) 1:2 (available from Rohm Pharma GmbH under the tradename EUDRAGIT S) and poly(methacrylic acid, methyl methacrylate) 1:1 (available from Rohm Pharma GmbH under the tradename EUDRAGIT L).

In one embodiment the pharmaceutically active agent is coated with a combination of a water insoluble film forming polymer (such as but not limited to cellulose acetate or ethylcellulose) and a water soluble polymer (such as but not limited to povidone, polymethacrylic co-polymers such as those sold under the tradename Eudragit E-100 from Rohm America, and hydroxypropylcellulose). In this embodiment, the ratio of water insoluble film forming polymer to water soluble polymer is from about 50 to about 95 percent of water insoluble polymer and from about 5 to about 50 percent of water soluble polymer, and the weight percent of the coating by weight of the coated taste-masked particle is from about 5 percent to about 40 percent. In one embodiment, the coating which is used in the coated particle of the pharmaceutically active agent is substantially free of a material (such as polyethylene glycol) which melts below 85° C., in order to prevent damage to the integrity of the coating during the RF heating step.

In one embodiment, one or more pharmaceutically active agents or a portion of the pharmaceutically active agent may be bound to an ion exchange resin for the purposes of taste-masking the pharmaceutically active agent or delivering the active in a modified release manner.

In one embodiment, the pharmaceutically active agent is capable of dissolution upon contact with a fluid such as water, stomach acid, intestinal fluid or the like. In one embodiment, the dissolution characteristics of the pharmaceutically active agent within the tablet meets USP specifications for immediate release tablets including the pharmaceutically active agent. For example, for acetaminophen tablets, USP 24 specifies that in pH 5.8 phosphate buffer, using USP apparatus 2 (paddles) at 50 rpm, at least 80% of the acetaminophen contained in the tablet is released there from within 30 minutes after dosing, and for ibuprofen tablets, USP 24 specifies that in pH 7.2 phosphate buffer, using USP apparatus 2 (paddles) at 50 rpm, at least 80% of the ibuprofen contained in the tablet is released there from within 60 minutes after dosing. See USP 24, 2000 Version, 19-20 and 856 (1999). In another embodiment, the dissolution characteristics of the pharmaceutically active agent are modified: e.g. controlled, sustained, extended, retarded, prolonged, delayed and the like.

In one embodiment, the particle size of the pharmaceutically active agent causes more void spaces to be present in the tablet, wherein a higher particle size of the pharmaceutically active agent subsequently requires a lower level of binder. In one embodiment, wherein the pharmaceutically active agent or coated pharmaceutically active agent(s) is greater than 50% of the blend by weight of the powder blend/tablet and the mean particle size of the carbohydrate is greater than 100 microns, the binder is from about 10 to about 30 percent by weight of the powder blend/tablet. In one embodiment, wherein the mean particle size of the powder blend is between about 100 microns and about 300 microns, then binder is from about 10 to about 20 percent by weight of the powder blend/tablet.

The melting point of the pharmaceutically active agent can have an impact on the temperature used during the heating step and the type of binder used. In one embodiment, the melting point of the binder is less than the melting point of the pharmaceutically active agent. In another embodiment, the melting point of the pharmaceutically active agent is the same or lower than the melting point of the binder, in which case during the fusing or heating step, both the pharmaceutically active agent and the binder may melt and create a eutectic or various bridges of the pharmaceutically active agent and binder between the other materials in the tablet form upon cooling. In one embodiment, the heating temperature is above the melting point of the binder and below the melting point of the pharmaceutically active agent. In one embodiment wherein ibuprofen is the pharmaceutically active agent, the binder is heated from about 30° C. to about 60° C. In one embodiment, the pharmaceutically active agent is the binder.

In one embodiment, the pharmaceutically active agent is in the form of a particle that is coated with the binder.

The susceptibility to RF energy of the pharmaceutically active agent (e.g., to melt or degrade) can have an impact on the type of energy and/or temperature used during the heating step as well as the type of the binder used.

In one embodiment, the processing of the tablet is free of a wet or hot melt granulation step. In this embodiment, the materials are directly blended prior to the addition of heat. In one embodiment, the materials are directly blended and compressed prior to the addition of heat.

### Energy Application to Powder Blend

The process includes the step of applying energy to a powder blend for a sufficient period of time to activate the binder(s) within the powder blend. Various forms of energy may be used in the process to activate the binder. Suitable sources of energy include but are not limited to convection, radio frequency, microwave, UV light, infrared, induction, laser light, and ultrasonic sound.

In one embodiment, the binder is a meltable material (e.g., an RF-meltable binder) having a melting point of from about 40° C. to about 140° C. and the powder blend is exposed to the energy for a sufficient period of time to melt or soften the meltable material.

In one embodiment, radiofrequency energy (“RF-energy”) is used. Radiofrequency heating generally refers to heating with electromagnetic field at frequencies from about 1 MHz to about 100 MHz. In one embodiment of the present invention, the RF-energy is within the range of frequencies from about 1 MHz to about 100 MHz (e.g., from about 5 MHz to 50 MHz, such as from about 10 MHz to about 30 MHz). In one embodiment, the RF-energy is used to heat the binder (e.g., either directly when the binder is a RF-meltable binder or indirectly when the binder is not a RF meltable binder but is heated by a RF-heatable ingredient within the powder blend). The type and amount of binder, and the amount of RF energy used can determine the hardness and/or type of tablet whether an oral disintegrating tablet or a soft chewable tablet is manufactured. RF energy generators are well known in the art. Examples of suitable RF generators include, but are not limited to, COSMOS Model C10X16G4 (Cosmos Electronic Machine Corporation, Farmingdale, N.Y.). In one embodiment the RF energy is combined with a second source of heat including but not limited to infrared, induction, or convection heating. In one embodiment, the addition of the second source of heat is particularly useful with a secondary non-RF-meltable binder present in the powder blend.

In one embodiment, microwave heating generally refers to heating with electromagnetic field at frequencies from about 100 MHz to about 300 GHz. In one embodiment of the present invention, the RF-energy is within the range of frequencies from about 500 MHz to about 100 GHz (e.g., from about 1 GHz to 50 GHz, such as from about 1 GHz to about 10 GHz). The microwave energy is used to heat the binder (e.g., either directly when the binder is susceptible to microwave energy (“microwave meltable binder”) or indirectly when the meltable binder is not a microwave meltable binder but is heated by a microwave heatable ingredient within the powder blend.

In one embodiment, the binder is water-activating binding material and the powder blend further comprises a water-containing material. In a further embodiment, the powder blend is exposed to the energy for a sufficient period of time to heat the water-containing material above its dehydration temperature and such released water activates the water-activating binding material.

In one embodiment the powder blend is heated as a unitary mass of material, wherein the mass is at least two times the size of a single dosage form, e.g. 10 times, e.g. 100 times or more of the mass of a single dosage form. In one embodiment, the powder blend is heated as a mass in a cylinder, tube, sheet or other chamber.

In one embodiment, the energy (e.g., RF energy) is applied for a sufficient time to activate (e.g., soften and melt) substantially all (e.g., at least 90%, such as at least 95%, such as all) of the binder within the powder blend.

In the embodiment where the powder blend is heated utilizing RF-energy, the electrodes are incorporated into a chamber holding the powder blend (e.g., a cylinder, walled-sheet, or other chamber). In one embodiment, the chamber is constructed of a conductive metal. In one embodiment, the chamber has portions which are constructed of non-conductive, insulative material. In the embodiment where the powder blend is heated utilizing conductive heating, the chamber may be constructed of a thermally conductive metal. In one embodiment, the chamber has an insert which is non-conductive where the body of the chamber is conductive. In one embodiment, the insert comprises a surface area which is less than that of the chamber. The conductive material may be comprised of any material which is conductive to RF-energy, including but not limited to aluminum, copper, iron, zinc, nickel and mixtures and alloys thereof. The non-conductive material may be comprised of a non-conductive solid material including but not limited to plastics and Teflon®. In one embodiment, the chamber has at least one electrode embedded into the walls of the cylinder or walled sheet. The electrode may be surrounded by non-conductive material wherein the electrode is the only conductive wall portion exposed to the power blend. In one embodiment, the powder blend is tamped prior to the addition of RF-energy.

In one embodiment, one chamber contains the powder blend and it is placed into a separate chamber (e.g., an oven) for the addition of energy. In another embodiment, the chamber containing the powder blend has additional heating elements incorporated into the chamber.

### Forming the Tablet

After the application of energy, the powder blend may optionally be cooled (e.g., actively cooled or allowed to cool) prior to forming a predetermined amount of the energy-applied powder blend into the tablet.

The tablets may be formed by various means.

In one embodiment, the shape of the tablet (e.g., tablet shape) is pre-formed by light compaction of the energy-applied powder blend. In one embodiment, the powder blend is fed into the tablet die of an apparatus that applies pressure to form the tablet shape (e.g., by light compaction such as tamping). Any suitable compacting apparatus may be used, including, but not limited to, a conventional unitary or rotary tablet press. In one embodiment, the tablet shape may be formed by compaction using a rotary tablet press (e.g., such as those commercially available from Fette America Inc., Rockaway, N.J. or Manesty Machines LTD, Liverpool, UK).

In one embodiment, the tablet is extruded from a cylinder after forming a mass by RF-energy, and the mass is cut into individual tablets as it is extruded from the cylinder utilizing a cutting apparatus such as a reciprocating or spinning blade. In another embodiment, the mass of powder to sintered into a unified sheet utilizing RF-energy and cut utilizing punches or dies in the dimension of a tablet. In another embodiment, the tablet is cut from the sintered powder mass utilizing a laser or water cutting tool.

In one embodiment, to obtain desired attribute of an orally transformable tablet, the tablet's construction may be highly porous, use a minimal amount of binder, and/or have a low density (e.g., to allow the tablet to collapse in the oral cavity). Such tablets, therefore, are somewhat fragile and soft. In a preferred embodiment, a minimum of tamping/compaction force is desired to achieve the orally transformable property (low density). Experiments have determined that low force compaction without application of energy to the powder blend produced very fragile tablets that could not withstand the forces of material handling required in manufacturing.

In one embodiment, the compaction step (e.g., tamping) which occurs prior to the addition of the energy utilizes a compaction force which is less than the force required to compress a chewable or swallowable tablet. In one embodiment, the compaction force is less than about 1000 pounds per square inch (e.g., less than about 500 pounds per square inch, such as less than 200 pounds per square inch, such as less than 50 pounds per square inch). In one embodiment, the energy is applied while the powder blend is under such force.

In one embodiment, the compaction step occurs in an indexed manner, where one set of tablets are compacted simultaneously, before rotating to another indexing station. In one embodiment, the compaction step occurs at a single indexing station and the application of energy occurs at a separate indexing station. In another embodiment, a third indexing station is present wherein the ejection of the tablet or multiple tablets occurs, wherein the lower forming tool is raised up through and up to the surface of the die. In another embodiment the compaction step is performed through the addition of air pressure or hydraulic cylinder to the top of the upper forming tools. In one embodiment multiple tablets are ejected simultaneously and separated from the surface of the indexing station and removed via a take-off bar.

In another embodiment, the tablet shape may be prepared by the compaction methods and apparatus described in United States Patent Application Publication No. 20040156902. Specifically, the tablet shape may be made using a rotary compression module including a fill zone, insertion zone, compression zone, ejection zone, and purge zone in a single apparatus having a double row die construction. The dies of the compression module may then be filled using the assistance of a vacuum, with filters located in or near each die. The purge zone of the compression module includes an optional powder blend recovery system to recover excess powder blend from the filters and return the powder blend to the dies.

In one embodiment, the tablet shape is prepared by the compaction methods and apparatus described in issued U.S. Pat. No. 6,767,200. Specifically, the tablet shape is made using a rotary compression module including a fill zone, compression zone, and ejection zone in a single apparatus having a double row die construction as shown in FIG. 6 therein. The dies of the compression module are preferably filled using the assistance of a vacuum, with filters located in or near each die.

The tablet shape may have one of a variety of different shapes. For example, the tablet shape may be shaped as a polyhedron, such as a cube, pyramid, prism, or the like; or may have the geometry of a space figure with some non-flat faces, such as a cone, truncated cone, triangle, cylinder, sphere, torus, or the like. In certain embodiments, a tablet shape has one or more major faces. For example, the tablet shape surface typically has opposing upper and lower faces formed by contact with the upper and lower forming tool faces (e.g., die punches) in the compaction machine. In such embodiments, the tablet shape surface typically further includes a “belly-band” located between the upper and lower faces, and formed by contact with the die walls in the compaction machine. A tablet shape/tablet may also be a multilayer. Applicants have found that sharp edges in the tooling used to make the tablets can cause arcing, and thus more rounded edges may be needed.

In one embodiment, the method of producing the tablet shape is substantially free of the use of solvents. In this embodiment, the powder blend is substantially free of solvents, and the manufacturing process (e.g., filling process into the die) is also substantially free of solvents. Solvents may include, but are not limited to, water, organic solvents such as but not limited to alcohols, chlorinated solvents, hexanes, or acetone; or gaseous solvents such as but not limited to nitrogen, carbon dioxide or supercritical fluids.

In one embodiment a vibratory step is utilized (e.g., added after filling of the powder blend but prior to the heating or fusing step, in order to remove air from the powder blend). In one embodiment a vibration with the frequency from about 1 Hz to about 50 KHz is added with amplitude from 1 micron to 5 mm peak-to-peak to allow for the flowable powder blend to settle into the cavity of a the die platen (“forming cavity”).

In one embodiment, the mass of energy-applied powder blend is cut to form the tablet. In one embodiment, the mass is then removed from a chamber (e.g. pushed or extruded) or cut within the chamber prior to removal. In one embodiment, the form is cut in a sequential fashion (e.g., as the mass is extruded from a cylinder). In another embodiment, multiple forms are cut simultaneously from the mass. The cutting apparatus and method may be in the form of a physical cutting apparatus such as a blade, a reciprocating blade or a circular or rotating blade. The cutting apparatus and method may be performed using a chemical or laser cutting apparatus.

Inserts within Tablet

In one embodiment, an insert is incorporated into the tablet. Examples include solid compressed forms or beads filled with a liquid composition.

In one embodiment the pharmaceutically active agent is in the form of a gel bead, which is liquid filled or semi-solid filled. The gel bead(s) are added as a portion of the powder blend. In one embodiment, the tablet of this invention has the added advantage of not using a strong compaction step, allowing for the use of liquid or semisolid filled particles or beads which are deformable since they will not rupture following the tablet forming step. These bead walls may contain gelling substances such as: gelatin; gellan gum; xanthan gum; agar; locust bean gum; carrageenan; polymers or polysaccharides such as but not limited to sodium alginate, calcium alginate, hypromellose, hydroxypropyl cellulose and pullulan; polyethylene oxide; and starches. The bead walls may further contain a plasticizer such as glycerin, polyethylene glycol, propylene glycol, triacetin, triethyl citrate and tributyl citrate. The pharmaceutically active agent may be dissolved, suspended or dispersed in a filler material such as but not limited to high fructose corn syrup, sugars, glycerin, polyethylene glycol, propylene glycol, or oils such as but not limited to vegetable oil, olive oil, or mineral oil.

### Multi-Layer Tablet

In certain embodiments, the tablet includes at least two layers, e.g., with different types and/or concentrations of binders and/or other ingredients or different concentrations of pharmaceutically active agents. In one embodiment, the tablet includes two layers, one layer having orally disintegrating properties and another layer being chewable or swallowable. In one embodiment, one layer has a binder and another layer does not have a binder. In one embodiment one layer is compacted at higher compaction force versus the other layer. In one embodiment, both layers contain same amount of binder, but have different amount of pharmaceutically active agents and/or other excipients. In one embodiment, all properties of the two layers are identical but the colors of the two layers are different.

### Effervescent Couple

In one embodiment, the powder blend further contains one or more effervescent couples. In one embodiment, effervescent couple contains one member from the group consisting of sodium bicarbonate, potassium bicarbonate, calcium carbonate, magnesium carbonate, and sodium carbonate, and one member selected from the group consisting of citric acid, malic acid, fumaric acid, tartaric acid, phosphoric acid, and alginic acid.

In one embodiment, the combined amount of the effervescent couple(s) in the powder blend/tablet is from about 2 to about 20 percent by weight, such as from about 2 to about 10 percent by weight of the total weight of the powder blend/tablet.

### Orally Disintegrating Tablet

In one embodiment, the tablet is designed to disintegrate in the mouth when placed on the tongue in less than about 60 seconds, e.g. less than about 45 seconds, e.g. less than about 30 seconds, e.g. less than about 15 seconds.

In one embodiment, the tablet meets the criteria for Orally Disintegrating Tablets (ODTs) as defined by the draft Food and Drug Administration guidance, as published in April, 2007. In one embodiment, the tablet meets a two-fold definition for orally disintegrating tablets including the following criteria: 1) that the solid tablet is one which contains medicinal substances and which disintegrates rapidly, usually within a matter of seconds, when placed upon the tongue and 2) be considered a solid oral preparation that disintegrates rapidly in the oral cavity, with an in vitro disintegration time of approximately 30 seconds or less, when based on the United States Pharmacopeia (USP) disintegration test method for the specific medicinal substance or substances.

### Additional Edible Portion

In one embodiment, the tablet is contained next to another edible form. In one embodiment, this edible form is a hard candy or compressed ring that holds the powder blend.

In one embodiment, the outer hard candy form may be made using uniplast rolling or roping and subsequent cutting and stamping, as well as depositing into molds. The hard candy portion contains one or more sugars selected from the group consisting of isomalt, sucrose, dextrose, corn syrup, lactitol, and lycasin. In one embodiment, the hard candy portion contains at least 50% (such as at least 75%, such as at least 90%) by weight of such sugar(s).

In one embodiment, the outer edible form contains a pharmaceutically active agent and the inner tablet contains a second portion of the same pharmaceutically active agent that is in the outer edible form. In one embodiment, the outer edible form contains a pharmaceutically active agent and the inner tablet contains a different pharmaceutically active agent than that in the outer edible form. In one embodiment, the outer edible form disintegrates at a rate of at least 10 times, such as at least 20 times, the rate of the inner tablet. The first and second portions can be the same or different.

In one embodiment, the tablet having an outer edible form and an inner tablet is coated with an immediate release sugar coating or film coating. In one embodiment, to produce such a tablet, the step following the fusing (heating) and subsequent cooling of the tablet would involve further sugar or film coating in a coating pan.

### Hardness/Density of Tablet

In one embodiment, the tablet is prepared such that the tablet is relatively soft (e.g., capable of disintegrating in the mouth or being chewed). In one embodiment, the hardness of the tablet is preferably less than about 3 kiloponds per square centimeter (kp/cm²) (e.g., less than about 2 kp/cm², such as less than about 1 kp/cm²).

Hardness is a term used in the art to describe the diametral breaking strength as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as tablet tensile strength. A general discussion of tablet hardness testing is found in Leiberman et al., Pharmaceutical Dosage Forms—Tablets, Volume 2, 2.sup.nd ed., Marcel Dekker Inc., 1990, pp. 213-217, 327-329.

A more preferred test for hardness of the tablet of the present invention relies upon a Texture Analyzer TA-XT2i that is fitted with a 7 millimeter diameter flat faced probe and setup to measure and report compression force in grams. The probe moves at 0.05 millimeters per second to a depth of penetration of 2 millimeters. The maximum compression force is recorded. In one embodiment, the measured forces recorded for tablets made in accordance with the present invention are less than 10,000 grams (e.g., less than about 1000 grams, such as less than about 700 grams. In one embodiment, the measured forces recorded for tablets made in accordance with the present invention ranges from about 100 grams to about 6000 grams, such as from about 100 grams to about 1000 grams, such as from about 75 grams to about 700 grams) with a deviation of ±50 grams. In another embodiment the measured forces recorded for tablets is less than 700 grams.

In one embodiment, the density of the tablet is less than about 2 g/cc (e.g., less than about 0.9 g/cc, such as less than about 0.8 g/cc, such as less than about 0.7 g/cc).

### Tablets Coatings

In one embodiment, the tablet includes an additional outer coating (e.g., a translucent coating such as a clear coating) to help limit the friability of the tablet. Suitable materials for translucent coatings include, but are not limited to, hypromellose, hydroxypropylcellulose, starch, polyvinyl alcohol, polyethylene glycol, polyvinylalcohol and polyethylene glycol mixtures and copolymers, and mixtures thereof. Tablets of the present invention may include a coating from about 0.05 to about 10 percent, or about 0.1 to about 3 percent by weight of the total tablet.

### Surface Treating of the Tablet

In one embodiment, the surface of the tablet is further treated with energy (e.g., convection, infrared, or RF energy) to soften or melt the material on the surface of the tablet and then cooled or allowed to cool to further smooth the texture, enhance the gloss of surface of the tablet, limit the friability of the tablet, and/or provide a mark for identification. In one embodiment, the surface of the tablet is further exposed to infrared energy wherein the majority (at least 50 percent, such as least 90 percent, such as at least 99 percent) of the wavelength of such infrared energy is from about 0.5 to about 5 micrometers such as from about 0.8 to about 3.5 micrometers (e.g., by use of a wavelength filter). In one embodiment, the infrared energy source is a quartz lamp with a parabolic reflector (e.g., to intensify the energy) and a filter to remove unwanted frequencies. Examples of such infrared energy sources include the SPOT IR 4150 (commercially available from Research, Inc., Eden Prairie, Minn.).

### Use of Tablet

The tablets may be used as swallowable, chewable, or orally disintegrating tablets to administer the pharmaceutically active agent.

In one embodiment, the present invention features a method of treating an ailment, the method including orally administering the above-described tablet wherein the tablet includes an amount of the pharmaceutically active agent effective to treat the ailment. Examples of such ailments include, but are not limited to, pain (such as headaches, migraines, sore throat, cramps, back aches and muscle aches), fever, inflammation, upper respiratory disorders (such as cough and congestion), infections (such as bacterial and viral infections), depression, diabetes, obesity, cardiovascular disorders (such as high cholesterol, triglycerides, and blood pressure), gastrointestinal disorders (such as nausea, diarrhea, irritable bowel syndrome and gas), sleep disorders, osteoporosis, and nicotine dependence.

In one embodiment, the method is for the treatment of an upper respiratory disorder, wherein the pharmaceutically active agent is selected from the group of phenylephrine, cetirizine, loratadine, fexofenadine, diphenhydramine, dextromethorphan, chlorpheniramine, chlophedianol, and pseudoephedrine.

In this embodiment, the “unit dose” is typically accompanied by dosing directions, which instruct the patient to take an amount of the pharmaceutically active agent that may be a multiple of the unit dose depending on, e.g., the age or weight of the patient. Typically the unit dose volume will contain an amount of pharmaceutically active agent that is therapeutically effective for the smallest patient. For example, suitable unit dose volumes may include one tablet.

### EXAMPLES

Specific embodiments of the present invention are illustrated by way of the following examples. This invention is not confined to the specific limitations set forth in these examples.

### Example 1

### Blend for a Sintered ODT Using Polymer Coated Active Ingredient

The powder blend of Table 1 was manufactured as follows.

TABLE 1
Powder Blend Formulation:

| | % Weight/ | |
|---|---|---|
| Granulation Blend | weight | Mg/Tablet |
| Dextrose Monohydrate | 38.08 | 415.0 |
| Polymer Coated | 50.92 | 555.56 |
| Acetaminophen¹ | | |
| Polyethylene Glycol | 5.96 | 65.0 |
| 4000 | | |
| Tapioca Maltodextrin | 3.67 | 40.0 |
| Red Colorant | 0.01 | 0.075 |
| Sucralose USP | 0.27 | 3.0 |
| Polyethylene Oxide | 0.46 | 5.0 |
| Mint Flavor | 0.69 | 7.5 |
| Total | 100.0 | 1091.14 |

1: Commercially Available from the Eurand Corporation

First, the sucralose, colorant, and flavor are placed together into a 500 cc sealable plastic bottle. The resulting mixture is then blended end-over-end manually for approximately 2 minutes. The resulting mixture and the coated acetaminophen, dextrose monohydrate, and polyethylene oxide are added to another 500 cc sealable plastic bottle and mixed end-over-end manually for approximately 5 minutes. Lastly, the polyethylene glycol 4000 and the tapioca maltodextrin is added to blend and mixed for approximately 3 minutes.

### Example 2

### Sintering of the ODT Containing Polymer Coated Active Ingredient within a Cylinder

A portion of the powder blend from Example 1 is placed into a plastic cylinder that is approximately 1.1″ in diameter, 0.175″ thick, and 50 cm in length. The cylinder containing the blend is placed between the electrodes of a RF heating unit, wherein the electrodes are incorporated into the walls of the cylinder a distance of approximately 1 cm. The cylinder is energized for 5 seconds. The sintered tablet blend is extruded from the cylinder and simultaneously cut using a metal blade into discs of 1091.14 mg per tablet. The tablets are subsequently printed with identification of “AP” and “500”.

### Example 3

### Sintering of the ODT Containing Polymer Coated Active Ingredient in the Shape of a Sheet

A portion of the powder blend from Example 1 is placed into a plastic sheet having side-walls. The sheet is approximately 30 cm×30 cm, with side-wall height of approximately 2.5 cm. The powder blend is layered onto the sheet at a height of approximately 0.75 cm. The sheet containing the powder blend is then placed between the electrodes of a RF heating unit, wherein the electrodes are incorporated into the walls of the metal sheet a distance of approximately 1 cm. The sheet is energized for 5 seconds. The sintered, formed blend is cut into individual dosage forms using a metal cutting die into discs of 1091.14 mg per tablet. The tablets are subsequently printed with identification of “AP” and “500”.

### Example 4

### Sintering the ODT in a Chamber and Forming in a Die

A portion of the powder blend from Example 1 is placed into a stainless steel cylinder equipped with a screw-mixing blade and a portal at the bottom of the cylinder. The cylinder has a diameter of about 5 cm and height of about 10 cm. The metal cylinder containing the powder blend is heated using convection by placing into an oven at 120 degrees Celsius for 15 minutes.

The cylinder is then removed from the oven, the mixer is activated. While the powder blend is still warm, the portal is opened while the screw mixer is turning at 10 RPM and the warm powder blend fed into a die pre-cooled at 20 degrees Celsius. The tablet is formed within the die using an upper and lower punch at a force less than 1 KP. The resulting tablets are produced at 1091.14 mg per tablet, ejected, and allowed to cool at room temperature for at least 5 minutes. The tablets are subsequently printed with identification of “AP” and “500”.

### Example 5

### Blend for a Sintered ODT Containing Hot-Melt Coated Active Ingredient

### Part A: Preparation of Hot-Melt Coated Ibuprofen

2000 g of ibuprofen crystals (particle size grade 110 μm) is charged into a Glatt fluid bed GPCG 5/9 coater equipped with a Wurster Insert. Glyceryl palmitostearate, commercially available from the Gattefosse Corporation (Saint-Priest, France) as Precirol® ATO, is placed into a suitable stainless steel container and melted at 70 degrees Celsius until it is completely melted. The melted material is sprayed onto the ibuprofen at a spray rate of approximately 20 g/minute while fluidizing at a product temperature of approximately 30 degrees Celsius. The coated ibuprofen is then discharged from the unit.

### Part B: Powder Blend Formulation

The powder blend of Table 2 is manufactured as follows:

TABLE 2
Powder Blend Formulation:

| | % Weight/ | |
|---|---|---|
| Granulation Blend | weight | Mg/Tablet |
| Dextrose Monohydrate | 53.85 | 410.0 |
| Hot-Melt Coated | 37.53 | 285.71 |
| Ibuprofen (70%) from | | |
| Example 5, Part A | | |
| Polyethylene Glycol | 2.63 | 20.0 |
| 4000 | | |
| Tapioca Maltodextrin | 4.60 | 35.0 |
| Red Colorant | 0.01 | 0.075 |
| Sucralose USP | 0.26 | 2.0 |
| Polyethylene Oxide | 0.39 | 3.0 |
| Mint Flavor | 0.72 | 5.5 |
| Total | 100.0 | 761.29 |

First, the sucralose, colorant, and flavor are placed together into a 500 cc sealable plastic bottle. The resulting mixture is then blended end-over-end manually for approximately 2 minutes. The resulting mixture and the coated ibuprofen, dextrose monohydrate, and polyethylene oxide are added to another 500 cc sealable plastic bottle and mixed end-over-end manually for approximately 5 minutes. Lastly, the polyethylene glycol 4000 and the tapioca maltodextrin is added to blend and mixed for approximately 3 minutes.

### Example 6

### Sintering of the ODT Containing a Hot Melt Coated Active Ingredient within a Cylinder

A portion of the powder blend from Example 5 is placed into a plastic cylinder that is approximately 1.1″ in diameter, 0.175″ thick, and 50 cm in length. The cylinder containing the blend is placed between the electrodes of a RF heating unit, wherein the electrodes are incorporated into the walls of the cylinder a distance of approximately 1 cm. The cylinder is energized for 5 seconds. The sintered tablet blend is extruded from the cylinder and simultaneously cut using a metal blade into discs of 1091.14 mg per tablet. The tablets are subsequently printed with identification of “AP” and “500”.

### Example 7

### Sintering of the ODT Containing a Hot Melt Coated Active Ingredient in the Shape of a Sheet

A portion of the powder blend from Example 5 is placed into a plastic sheet having side-walls. The sheet is approximately 30 cm×30 cm, with side-wall height of approximately 2.5 cm. The powder blend is layered onto the sheet at a height of approximately 0.75 cm. The sheet containing the powder blend is then placed between the electrodes of a RF heating unit, wherein the electrodes are incorporated into the walls of the metal sheet a distance of approximately 1 cm. The sheet is energized for 5 seconds. The sintered, formed blend is cut into individual dosage forms using a metal cutting die into discs of 1091.14 mg per tablet. The tablets are subsequently printed with identification of “AP” and “500”.

### Example 8

### Sintering the ODT in a Chamber and Forming in a Die

A portion of the powder blend from Example 5 is placed into a stainless steel cylinder equipped with a screw-mixing blade and a portal at the bottom of the cylinder. The cylinder has a diameter of about 5 cm and height of about 10 cm. The metal cylinder containing the powder blend is heated using convection by placing into an oven at 120 degrees Celsius for 15 minutes.

The cylinder is then removed from the oven, the mixer is activated. While the powder blend is still warm, the portal is opened while the screw mixer is turning at 10 RPM and the warm powder blend fed into a die pre-cooled at 20 degrees Celsius. The tablet is formed within the die using an upper and lower punch at a force less than 1 KP. The resulting tablets are produced at 1091.14 mg per tablet, ejected, and allowed to cool at room temperature for at least 5 minutes. The tablets are subsequently printed with identification of “AP” and “500”.

### Example 9

### Preparation of a Acetaminophen Powder Blend Utilizing a Water Activated Binder

The acetaminophen powder blend for an orally disintegrating tablet, containing the ingredients of Table 3, is manufactured as follows:

TABLE 3
Coated APAP Powder Blend Formulation

| Ingredient | G/Batch | Mg/Tablet |
|---|---|---|
| Dextrose Monohydrate | 6.14 | 588.78 |
| Maltodextrin | 0.6 | 54 |
| Sucralose NF | 0.06 | 5.4 |
| D&C Yellow #10 AI Lake | 0.01 | 0.54 |
| Coated Acetaminophen (91%)* | 1.94 | 174.78 |
| Peppermint | 0.1 | 9 |
| Disodium H Phosphate | 0.75 | 67.5 |
| Dodecahydrate USP** | | |
| Total | 10 | 0 |

*Commercially Available from Eurand in Vidalia, OH
**Commercially available from the Dow Corporation in Midland, MI

Each ingredient is screened through a 30 mesh screen, placed together into a 500 cc plastic bottle, and mixed end-over-end for 5 minutes.

### Example 10

### Sintering of the ODT Containing a Hot Melt Coated Active Ingredient within a Cylinder

A portion of the powder blend from Example 9 is placed into a plastic cylinder that is approximately 1.1″ in diameter, 0.175″ thick, and 50 cm in length. The cylinder containing the blend is placed between the electrodes of a RF heating unit, wherein the electrodes are incorporated into the walls of the cylinder a distance of approximately 1 cm. The cylinder is energized for 5 seconds. The sintered tablet blend is extruded from the cylinder and simultaneously cut using a metal blade into discs of 1091.14 mg per tablet. The tablets are subsequently printed with identification of “AP” and “500”.

### Example 11

### Sintering of the ODT Containing a Hot Melt Coated Active Ingredient in the Shape of a Sheet

A portion of the powder blend from Example 9 is placed into a plastic sheet having side-walls. The sheet is approximately 30 cm×30 cm, with side-wall height of approximately 2.5 cm. The powder blend is layered onto the sheet at a height of approximately 0.75 cm. The sheet containing the powder blend is then placed between the electrodes of a RF heating unit, wherein the electrodes are incorporated into the walls of the metal sheet a distance of approximately 1 cm. The sheet is energized for 5 seconds. The sintered, formed blend is cut into individual dosage forms using a metal cutting die into discs of 1091.14 mg per tablet. The tablets are subsequently printed with identification of “AP” and “500”.

### Example 12

### Sintering the ODT in a Chamber and Forming in a Die

A portion of the powder blend from Example 9 is placed into a stainless steel cylinder equipped with a screw-mixing blade and a portal at the bottom of the cylinder. The cylinder has a diameter of about 5 cm and height of about 10 cm. The metal cylinder containing the powder blend is heated using convection by placing into an oven at 120 degrees Celsius for 15 minutes.

The cylinder is then removed from the oven, the mixer is activated. While the powder blend is still warm, the portal is opened while the screw mixer is turning at 10 RPM and the warm powder blend fed into a die pre-cooled at 20 degrees Celsius. The tablet is formed within the die using an upper and lower punch at a force less than 1 KP. The resulting tablets are produced at 1091.14 mg per tablet, ejected, and allowed to cool at room temperature for at least 5 minutes. The tablets are subsequently printed with identification of “AP” and “500”.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A process for making tablets comprising one or more pharmaceutically active agent(s) and one or more binder(s), said method comprising the steps of (i) applying energy to a powder blend comprising said pharmaceutically active agent(s) and said binder(s) for a sufficient period of time to activate said binder(s) within said powder blend and (ii) forming a predetermined amount of said energy-applied powder blend into said tablets, wherein said tablets have a density less than 0.8 g/cc and said tablets disintegrate in the mouth when placed on the tongue in less than about 30 seconds.

2. The process of claim 1, wherein said binder is a meltable material having a melting point of from about 40° C. to about 140° C. and said powder blend is exposed to said energy for a sufficient period of time to melt or soften said meltable material.

3. The process of claim 2, wherein said binder is an RF-meltable binder and said powder blend is exposed to RF energy for a sufficient period of time to melt or soften said meltable material.

4. The process of claim 3, wherein said radiofrequency energy has a frequency of from about 1 MHz to 100 MHz.

5. The process of claim 1, wherein said binder is water-activating binding material, said powder blend further comprises a water-containing material, and said powder blend is exposed to said energy for a sufficient period of time to heat the water-containing material above its dehydration temperature.

6. The process of claim 1, wherein said powder blend comprises from about 0.01 to about 30 percent, by weight, of said pharmaceutically active agent and from about 1 to about 30 percent, by weight, of said meltable binder.

7. The process of claim 1, wherein said binder is polyethylene glycol.

8. The process of claim 1, wherein said powder blend further comprises one or more carbohydrates selected from the group consisting of dextrose monohydrate, mannitol, erythritol, dextrose, lactose, sorbitol, isomalt, sucrose, dextrates and maltodextrins.

9. The process of claim 8, wherein said powder blend comprises from about 30 to about 95 percent, by weight, of said one or more carbohydrates.

10. The process of claim 1, wherein said powder blend has an average particle size of less than 500 microns.

11. A process of claim 1, wherein said energy-applied powder blend is cooled prior to forming said tablets.

12. A process of claim 1, wherein said tablets are formed within a tablet die.

13. A process of claim 1, wherein said energy-applied powder blend is cut to form said tablets.

14. The process of claim 1, wherein said tablets meet the criteria for Orally Disintegrating tablets as defined by the draft Food & Drug Administration guidance, as published April, 2007.

15. The process of claim 1, wherein said tablets have a hardness of less than 700 grams as measure using Texture Analyzer TA-XT2i that is fitted with a 7 millimeter diameter flat faced probe.

16. The process of claim 1, wherein the surface of said tablets are further exposed to infrared energy wherein the majority of the wavelength of said infrared energy from about 0.5 to about 5 micrometers.

17. A process for making tablets comprising applying radiofrequency energy to a powder blend comprising a pharmaceutically active agent and a RF-meltable binder for a sufficient period of time to soften or melt said RF-binder and forming a predetermined amount of said energy-applied powder blend into said tablets.

18. The process of claim 17, wherein said tablets have a density less than 0.8 g/cc.

19. The process of claim 15, wherein said tablets disintegrates in the mouth when placed on the tongue in less than about 30 seconds.

20. A tablet manufactured according to the process of claim 1.
